(19) United States
(12) Patent Application Publication
(10) Pub. No.: US 2017/0265786 A1
(43) Pub. Date: Sep. 21, 2017

(54) METHODOLOGY AND APPARATUS FOR DETERMINING PSYCHOACOUSTICAL THRESHOLD CURVES

(71) Applicant: Danmarks Tekniske Universitet, Kgs. Lyngby (DK)

(72) Inventors: Michal Fereczkowski, Kgs. Lyngby (DK); Ewen Neale MacDonald, Frederiksberg (DK); Torsten Dau, Copenhagen K (DK)

(73) Assignee: Danmarks Tekniske Universitet, KGS. Lyngby (DK)

(21) Appl. No.: 15/505,852

(22) PCT Filed: Sep. 18, 2015
(86) PCT No.: PCT/EP2015/071420

§ 371 Date: Feb. 22, 2017

(30) Foreign Application Priority Data
Sep. 25, 2014 (EP) 14186405.8
May 15, 2015 (EP) 15167840.6

(51) Int. Cl. A61B 5/12; A61B 5/00

(52) U.S. Cl. CPC A61B 5/123; A61B 5/7203

(57) ABSTRACT

The present invention relates in a first aspect to a method of determining a psychoacoustical threshold curve by selectively varying a first parameter and a second parameter of an auditory stimulus signal applied to a test subject/listener. The methodology comprises steps of determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve.

A series of auditory stimulus signals in accordance with the respective parameter pairs are presented to the listener through a sound reproduction device and the listener's detection of a predetermined attribute/feature of the auditory stimulus signals is recorded such that a stimuli path through the predetermined two-dimensional response space is traversed. The psychoacoustical threshold curve is computed based on at least a subset of the recorded parameter pairs.

The present invention relates in a first aspect to a method of determining a psychoacoustical threshold curve by selectively varying a first parameter and a second parameter of an auditory stimulus signal applied to a test subject/listener. The methodology comprises steps of determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve.

A series of auditory stimulus signals in accordance with the respective parameter pairs are presented to the listener through a sound reproduction device and the listener's detection of a predetermined attribute or feature of the auditory stimulus signals is recorded such that a stimuli path through the predetermined two-dimensional response space is traversed. The psychoacoustical threshold curve is computed based on at least a subset of the recorded parameter pairs.

### BACKGROUND OF THE INVENTION

Psychophysics is an area of science which uses mathematical tools to quantify psychological and physiological responses of humans to physical stimuli. Psychoacoustics is a part of psychophysics where sound is used as stimulus.

Due to individual differences in human listeners, it is necessary to run psychoacoustical experiments to determine individual thresholds. Often, experimenters are interested in determining how a detection threshold changes when a certain parameter of the sound stimulus is varied. In some cases the sound parameter can, in principle, be varied continuously. However, due to time limitations that are always present in experimental or clinical practice, only relatively few values of the sound parameter are selected and corresponding thresholds determined. Subsequently, interpolation/extrapolation techniques may be utilized in order to approximate the thresholds for untested parameter values to determine or compute a complete psychoacoustical threshold curve across a target range parameters.

A well-known example of such a psychoacoustical experiment, which is used in clinical practice, is so-called, Pure Tone Audiometry. In Pure Tone Audiometry the sound stimulus is a sinusoid with a fixed duration (usually 200 ms-500 ms). The parameter under investigation is the frequency of the sinusoid. Respective hearing thresholds are found for a predetermined number of test frequencies, for example six frequencies 250, 500, 1000, 2000, 4000 and 8000 Hz. Subsequently, a linear interpolation is used in the logarithmic domain to obtain the psychoacoustical threshold curve called “an audiogram”. It is worth noting that the psychoacoustical threshold curve is a collection of (e.g. 50%) thresholds estimated for varying parameter values.

A particular kind of psychoacoustical threshold curves of interest in audiological research is the so-called temporal masking curves (TMCs) as they may allow for diagnosis of the state of the inner ear (cochlea). The diagnosis of the state and sound processing capabilities of the inner ear is believed to be useful for numerous hearing aid fitting procedures for example to determine optimal and individualized dynamic range compressor characteristics of multi-band dynamic range compression systems of digital hearing instruments. However, current methods of determining such individualized temporal masking curves are generally time-consuming. One such current methodology is described in Nelson, D. A. (2001) “A new procedure for measuring peripheral compression in normal hearing and hearing-impaired listeners”, JASA, 110 (4), 2045-2064.

Hearing aid fitting in clinical practice must rely on a rapid and reliable methodology of determining the patients' hearing loss, via measurement of a suitable psychoacoustical threshold curve(s), to provide a satisfactory patient throughput and hence maintain profitability for the hearing aid dispenser or clinic. The speed of the procedure is also important to preserve the well-being of the patient and prevent that the reliability of the acquired hearing loss data are compromised by patient fatigue.

Consequently, new procedures, methodologies and audiological test apparatuses which allow rapid acquisition or determination of individual psychoacoustical threshold curves of patients' in connection with psychoacoustical experiments are highly desirable. The present methodologies allow rapid acquisition of individual psychoacoustical threshold curves, in particular TMCs, because of changing the way of sampling or traversing the parameter-response space. The present methodologies of determining psychoacoustical threshold curves are adapted to markedly reduce experimental time by increasing the proportion of time spent on presenting auditory stimulus signals in the vicinity of the sought after psychoacoustical threshold curve compared to current state-of the-art methods. This feature is able to significantly speed-up the determination of individual psychoacoustical threshold curves of listeners or test subjects such as hearing impaired individuals.

### SUMMARY OF THE INVENTION

A first aspect of the invention relates to a method of determining a psychoacoustical threshold curve by selectively varying a first parameter and a second parameter of an auditory stimulus signal applied to a test subject or listener, comprising steps of:

a) determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve,
b) instructing the listener to detect a predetermined attribute/feature of the auditory stimulus signal,
c) determining a first parameter pair comprising a value of the first parameter and a value of the second parameter where the first parameter pair is situated in the positive response region,
d) presenting a first auditory stimulus signal in accordance with the first parameter pair to the listener through a sound reproduction device and recording the listener's positive or negative detection of the predetermined attribute/feature of the first auditory stimulus signal,
e) presenting a subsequent auditory stimulus signal(s) to the listener in accordance with a subsequent parameter pair following a former parameter pair in a first path direction through the two dimensional response space; wherein the first path direction heads towards the a priori estimated placement of the psychoacoustical threshold curve,
f) recording the listener's positive or negative detection of the predetermined attribute/feature of the subsequent auditory stimulus signal(s) and repeat steps e) and f) until a reversal detection in accordance with a predetermined detection reversal criterion is fulfilled in the first path direction or until the two-dimensional boundary region is reached,
g) select a subsequent parameter pair following the former parameter pair in a second path direction, differing from the first path direction and its reverse, in the predetermined two-dimensional response space, wherein the second path direction heads towards the a priori estimated placement of the psychoacoustical threshold curve,
h) presenting a subsequent auditory stimulus signal in accordance with the subsequent parameter pair to the listener and recording the listener's positive or negative detection of the predetermined attribute/feature of the subsequent auditory stimulus signal,
i) repeat step h) until a reversal detection in accordance with the predetermined detection reversal criterion is fulfilled in the second path direction or until the two-dimensional boundary region is reached,
j) repeating steps e), f), g), h) and i) one or more times to traverse and record a stimuli path through the predetermined two-dimensional response space extending forth and back across the psychoacoustical threshold curve,
k) determining the psychoacoustical threshold curve based on at least a subset of the recorded parameter pairs indicating the stimuli path through the predetermined two-dimensional response space.

The skilled person will understand that the present method of determining psychoacoustical threshold curves can be applied to a wide range of psychoacoustical threshold curves by proper selection of the first and second parameters of the auditory stimulus signal such as temporal masking curves (TMCs), Growth of Masking (GOM) curves, modulation detection threshold curves, frequency selectivity tests such as a notched noise experiment, audiogram curves etc. The skilled person will furthermore understand that the present method of determining psychoacoustical threshold curves may be applied to both detection experiments and discrimination experiments by a proper construction of the auditory stimulus signal.

Furthermore, the skilled person will appreciate that the change from the first path direction to the second path direction, where the latter is different from the first path direction and the reverse thereof, in response to the compliance with the predetermined detection reversal criterion, has important methodological advantages. In particular, ensuring that most of the experimental time is spent on presenting auditory stimulus signals with first and second parameters in the vicinity of the sought after psychoacoustical threshold curve. Hence, by avoiding starting measurement of each individual threshold from a simple condition of the auditory stimulus signal, i.e. far away from the threshold condition, and avoiding travelling through a previously travelled return path in the first direction leading back across the psychoacoustical threshold curve, the number of auditory stimulus signals that need to be presented to the listener or patient in order to estimate the desired psychoacoustical threshold curve is minimized. This feature has several important benefits. One benefit is a marked reduction of the length or duration of the test procedure or methodology for determining the psychoacoustical threshold curve compared to prior art methodologies. This reduced test procedure duration allows the present methodology to be applied to hearing aid fitting in clinical practice as discussed above. The reduced duration of the present test methodology also improves the reliability and accuracy of the acquired listener responses, i.e. the positive or negative detections of the predetermined attribute or feature of the auditory stimulus signals, by reducing listener or patient fatigue.

The present methodology also reduces the amount of data concerning the listener's positive or negative detection responses which needs recording and storage in a memory of the audiological test apparatus or equipment. The reduction of recorded listener response data is caused by the more efficient sampling of the predetermined two-dimensional response space, i.e. only auditory stimulus signals with first and second parameters situated in the vicinity of the sought after psychoacoustical threshold curve need to be presented to the listener and corresponding detection responses stored. Moreover, in case of different listeners traversing a discrete grid within the two-dimensional boundary region, the list of possible parameter pairs is limited in length. This feature makes it possible to determine and store the set of auditory stimulus signals to be presented before experiments. This is advantageous when the audiological test apparatus or equipment has relatively low computational power, or, for other reasons (i.e. missing libraries of a programming language) is incapable of computing auditory stimulus signals. Another advantageous effect is that as the test continues, a test supervisor or clinician can monitor (e.g. via a suitable display) how the psychoacoustical threshold curve develops and therefore rapidly manually intervene at anomalous behavior of the listener. It is not possible to obtain this type of helpful real-time feedback for standard techniques where several point of the threshold curve must be measured before visualizing an approximated threshold curve.

The presence of the two-dimensional boundary region helps with limiting or controlling the dynamic range of the auditory stimulus signals in a way that does not lead to skipping of measurement paths when, or if, an upper or lower boundary limit is reached as in prior art standard, oscillating, adaptive threshold finding procedures. Apart from such practical consequences, the two-dimensional boundary region allows the auditory stimulus signals to be represented efficiently e.g. by 16 bit audio samples, which corresponds to about 96 dB dynamic range, without introducing audible quantization noise.

According to a preferred embodiment of the presently methodology of determining a psychoacoustical threshold curve, the second path direction extends substantially orthogonally to the first path direction in the predetermined two-dimensional response space. This feature leads to significant benefits when the first path direction is selected such that it corresponds to constant values of the second parameter and the second path direction is selected such that it corresponds to constant values of the first parameter. Hence, only one of the first parameter and second parameter of the auditory stimulus signal is varied between subsequent auditory stimulus presentations when traversing the predetermined two-dimensional response space. This feature is a significant advantage in numerous types of psychoacoustical threshold curve measurements because listener responses are generally more consistent when only one property of the presented auditory stimulus signal changes at a time. In temporal masking curve (TMC) applications of the present methodology, the first parameter may be a time gap between a masker tone and a probe tone of the each of the auditory stimulus signals. The first parameter values, e.g. time gap values, may be mapped along a first direction or axis of the two-dimensional response space. The second parameter may be a masker tone sound pressure level of the auditory stimulus signals. The second parameter, e.g. the masker tone sound pressure level, may be mapped along a second direction or axis of the two-dimensional response space, where the second direction is orthogonal or perpendicular to the first direction. Consequently, when moving in the first direction, which may be horizontal I, through the two-dimensional response space only the masker tone sound pressure is varied while time gap is kept essentially constant. When moving in the second direction, which may be vertical, through the two-dimensional response space only the time gap is varied while the masker tone sound pressure level is kept essentially constant.

In another embodiment, the predetermined two-dimensional response space comprises a predetermined two-dimensional parameter grid structure comprising a plurality of parameter pairs comprising respective values of the first and second parameters. The two-dimensional parameter grid structure may be used to select a discrete set of values of the first parameter and a discrete set of values of the second parameter before the test methodology is initiated. During execution of the present methodology (of determining the psychoacoustical threshold curve) all of the parameter pairs of the presented auditory stimulus signals may lie on the parameter grid structure. Hence, when stepping or jumping between in the first path direction or the second path direction from a given parameter pair to the subsequent parameter pair, both of these parameter pairs lie on respective grid points of the two-dimensional parameter grid structure. The step from the given parameter pair to the subsequent parameter pair may be between adjacent parameter pairs in the first direction or between adjacent parameter pairs in the second direction such that the subsequent parameter pair of each of steps e) and g) is placed adjacent to the former parameter pair on the two dimensional parameter grid structure. Alternatively, the step from the given parameter pair to the subsequent parameter pair may discard one or more intermediate grid point(s) with respective parameters pairs situated between the given parameter pair and subsequent parameter pair in the first direction or the second direction. In yet another embodiment, an adaptive step size may be applied such that the step size through the two-dimensional parameter grid structure varies depending on the listener's responses to so far presented auditory stimulus signals or depending on an estimated distance to the psychoacoustical threshold curve. Hence, the size of the steps through the predetermined two-dimensional response space may for example be smaller when a given parameter pair lies close to the a priori estimated placement of the psychoacoustical threshold curve than when the parameter pair is further away from estimated placement of the psychoacoustical threshold curve.

The skilled person will appreciate that the reversal detection under each of the steps f) and i) of the present methodology may be accepted immediately at the listener's first reversal detection in the current path direction or that more elaborate criteria may be applied to obtain further confidence in the validity of the initial or first detection reversal before changing path direction from the first to the second path direction or vice versa. Hence, the present methodology comprises a predetermined detection reversal criterion which must be fulfilled to accept the validity of a given detection reversal. According to the above-mentioned immediate accept of the listener's detection reversal the predetermined detection reversal criterion comprises
identifying an initial response reversal in the first path direction or an initial response reversal in the second path direction,
selecting the subsequent parameter pair in opposite direction, i.e. the first path direction or the second path direction as the case may be, of the former parameter pair, and
present the subsequent auditory stimulus signal in accordance with the subsequent parameter pair.

Various more sophisticated predetermined detection reversal criteria may lead to higher confidence in the validity of any particular reversal detection albeit at the expense of an increasing number of auditory stimuli presentations and therefore increasing test procedure time. An exemplary sophisticated detection reversal criterion comprises:
identifying an initial detection reversal in the first path direction or an initial detection reversal in the second path direction,
repeating the presentation of the auditory stimulus signal that led to the initial response reversal,
if the reversal detection is confirmed then proceed to step e) or step g) to proceed in an opposite path direction to a current path direction; and
if the reversal detection is denied then determine a subsequent parameter pair arranged in the same path direction as the former parameter pair, and present a subsequent auditory stimulus signal in accordance with the subsequent parameter pair. In this manner, the listener is forced to confirm his/hers initial detection reversal at least one more time with the same auditory stimulus signal before the current path direction is changed.

If the two-dimensional boundary region is reached during repetition of steps e) and f) above, or repetition of steps i) and h) above, corrective action is preferably taken because this incident may indicate erroneous listener responses for example caused by fatigue or lacking understanding of the detection task at hand. Various types of corrective actions may be taken depending on which corner, or which boundary limit of the two-dimensional boundary region, is reached as described in further detail below in connection with the appended drawings.

The psychoacoustical threshold curve may have various shapes such as either monotonically decreasing throughout the predetermined two-dimensional response space or monotonically increasing throughout the predetermined two-dimensional response space. The present methodology may also be applied to determine other shapes of the psychoacoustical threshold curve, such as concave or convex shapes, by appropriate adaptation of the stimuli path as discussed in further detail below with reference to the appended drawings.

A preferred embodiment of the invention is adapted to determine a temporal masking curve (TMC) as previously discussed. Hence, each of the auditory stimulus signals may comprise a masker tone and a probe/signal tone separated by a time gap; and the predetermined attribute/feature of each of the auditory stimulus signals is the probe/signal tone such that the listener's task is to detect and indicate the presence or absence of the probe tone. The first parameter of the auditory stimulus signals is associated with a signal property of the masker tone and the second parameter is associated with either a signal property of the probe tone or a property of the time gap. Various combinations of first and second parameters of the auditory stimulus signals may be selected for variation in connection with the determination or measurement of a temporal masking curve (TMC). In one embodiment, the first parameter of the auditory stimulus signals comprises a level of the masker tone and the second parameter of the auditory stimulus signals comprises the time gap between the masker tone and the probe tone such that the psychoacoustical threshold curve represents a temporal masking curve (TMC) of the test subject/listener. The signal characteristics of the auditory stimulus signal and the selection of the first and second parameters of the auditory stimulus signals are discussed in further detail below with reference to the appended drawings. The time gap values may be mapped along the first axis of the two-dimensional response space and levels of the masker tone may be mapped along the second axis, which preferably is orthogonal to the first axis, of the two-dimensional response space. The parameter pairs mapped to the two-dimensional response space preferably at least comprises: time gap values between 1 ms and 200 ms with predetermined linear or logarithmic gap spacing; and

masker level values between 10 dB SPL and 85 dB SPL with a predetermined linear or logarithmic level spacing. Various exemplary sets of time gap values and masker sound pressure levels in connection with the determination of temporal masking curves are discussed in further detail below with reference to the appended drawings.

Useful embodiments of the present methodology comprise a measurement of an audiogram of the listener prior to performing step a) of the present method of determining a psychoacoustical threshold curve. The range of values of each of the first and second parameters of the auditory stimulus signals may be derived from hearing loss data acquired during such an initial audiogram measurement of the listener. Likewise, hearing loss data acquired during the initial audiogram measurement may be used to derive upper and lower bounds or limits of the two-dimensional boundary region for the first parameter and upper and lower limits of the two-dimensional boundary region for the second parameter. This will ensure that the characteristics of the presented auditory stimulus signals are aligned with the hearing ability of the listener. This feature has numerous benefits such as ensuring that the sound pressure level of the auditory stimulus signal never exceeds the so-called uncomfortable level (UCL) of the listener and/or that the predetermined attribute or feature of the auditory stimulus signal lies above the listener's hearing threshold. This feature may be used to set the limits of the two-dimensional boundary region in a meaningful manner such that the characteristics of the presented auditory stimulus signals are adapted to the type of psychoacoustical threshold curve in question and to the listener's basic hearing abilities and/or comfort.

In connection with TMC determinations, the lower limit of the two-dimensional response space with respect to the level of the masker tone may for example be determined from the audiogram in an indirect manner. Initially, the fixed and pre-selected level of the probe tone is derived from the audiogram data of the listener. Thereafter, the lower limit of the level of the masker tone is derived from the selected fixed level of the probe tone for example between 2 and 20 dB SPL below the fixed level of the probe tone. The upper limit of the two-dimensional response space with respect to the level of the masker tone may also be determined based on the measured hearing loss of the listener, preferably the hearing loss at the frequency of the masker signal. The hearing loss at the frequency of the masker tone may be used to estimate the listener's UCL level at the frequency of the masker tone such that the upper limit of the two-dimensional response space with respect to the level of the masker tone is set to the estimated UCL level.

According to yet another embodiment of the present methodology of determining a psychoacoustical threshold curve, the listener is subjected to one or more so-called catch trials prior to performing step a) of the method or randomly placed between the non-catch stimuli presentations and response recordings according to steps e)-i). The catch trial comprises a presentation of one of more auditory stimulus signals where predetermined attribute/feature is absent. The one or more catch trials are often helpful by preventing the listener from adopting unwanted response strategies. The one or more catch trials are also helpful for the listener as a point of reference for the negative detection response.

According to another embodiment of the present methodology of determining a psychoacoustical threshold curve, the listener is presented with an auditory stimulus signal which comprises two or more stimuli (so-called intervals or alternative). Only one of these two or more stimuli has the predetermined attribute/feature, otherwise the two or more stimuli are identical. The listener's task is to detect which stimuli had the attribute, or if more than two stimuli are presented in a single auditory stimulus signal, which stimulus was different from the rest. This embodiment of the present methodology, where multiple (n) alternatives are given to the listener, is often designated n-alternative-forced choice or n-interval-forced choice. The skilled person will understand that the listener's positive detection or response is equivalent to a “correct” response in such multiple (n) alternative experiments. Likewise, the listener's negative detection or response (e.g. “inaudible/No”) is equivalent to “incorrect” response.

A second aspect of the present invention relates to a method of determining a basilar membrane input/output curve of a listener at one or several audiologically relevant test frequencies based on temporal masking curves. The method comprising steps of:

a) selecting a first test frequency,
b) applying the method of determining the temporal masking curve described above to the listener a first time where a frequency of the probe tone is equal to the first test frequency and set a frequency of the masker tone at least one-half octave lower than the first test frequency,
c) record and store in a data memory device a first temporal masking curve resulting from the first time of application of the method of determining the temporal masking curve,
d) applying the method of determining the temporal masking curve described above to the listener a second time where the frequency of the probe tone and the frequency of the masker tone are both substantially equal to the first test frequency,
e) record and store in the data memory device a second temporal masking curve resulting from the second time of application of the method of determining the temporal masking curve,
f) compute the listener's basilar membrane input/output curve at the first test frequency based on the first and second temporal masking curves.

The skilled person will appreciate that the above-mentioned method of determining the basilar membrane input/output curve at the first test frequency may be extended to one or more additional test frequencies of audiological relevance to investigate or characterize the listener's hearing loss in further detail. The characteristics of the listener's basilar membrane input/output curve may vary considerably with frequency. The first frequency, and any of the additional test frequencies, may lie between 100 Hz and 10 kHz for example one of more test frequencies selected from a group of {125 Hz, 250 Hz, 500 Hz, 1 kHz, 2 kHz, 4 kHz, 8 kHz}.

The skilled person will understand that the above-mentioned method of determining the listener's basilar membrane input/output curve on the basis of a preceding determination of the listener's temporal masking curves may be highly useful as an initial step of a hearing aid fitting procedure. In particular, the listener's basilar membrane input/output curve at a particular set of test frequencies of audiological relevance may be used to derive corresponding frequency band specific compression parameter settings of a multi-channel dynamic range compression function or algorithm of a hearing instrument. These compression parameter settings may for example be used to derive a compression ratio and compression threshold for some, or each, of the frequency bands of the multi-channel dynamic range compression function or algorithm.

A third aspect of the present invention relates to an audiological test apparatus for determining a psychoacoustical threshold curve by selectively varying a first parameter and a second parameter of an auditory stimulus signal applied to a test subject or listener, the apparatus comprising:

a programmable computer controlled by a test program comprising a plurality of executable program instructions or code,
a sound reproduction device such as headphones or earphones configured to apply auditory stimulus signals to the listener,
a response detector configured to detect and record listener responses to the presented auditory stimulus signals, and
a programmable sound generator configured to generate auditory stimulus signals in accordance with a plurality of signal parameters, wherein a processor of the test apparatus is configured to, by execution of the test program, execute steps of:
a) determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve,
b) optionally instructing the listener to detect a predetermined attribute/feature of the auditory stimulus signal,
c) determining a first parameter pair comprising a value of the first parameter and a value of the second parameter where the first parameter pair is situated in the positive response region,
d) presenting a first auditory stimulus signal in accordance with the first parameter pair to the listener through the sound reproduction device and recording the listener's positive or negative detection of the predetermined attribute/feature of the first auditory stimulus,
e) presenting a subsequent auditory stimulus signal(s) to the listener in accordance with a subsequent parameter pair arranged adjacent to a former parameter pair in a first path direction through the two dimensional response space; wherein the first path direction heads towards the a priori estimated placement of the psychoacoustical threshold curve,
f) recording the listener's positive or negative detection of the predetermined attribute/feature of the subsequent auditory stimulus signal(s) and repeat steps e) and f) until a reversal detection in accordance with a predetermined detection reversal criterion is fulfilled in the first path direction or until the two-dimensional boundary region is reached,
g) select a subsequent parameter pair following the former parameter pair in a second path direction, differing from the first path direction and its reverse, in the predetermined two-dimensional response space, wherein the second path direction heads towards the a priori estimated placement of the psychoacoustical threshold curve,
h) presenting a subsequent auditory stimulus signal in accordance with the subsequent parameter pair to the listener and recording the listener's positive or negative detection of the predetermined attribute/feature of the subsequent auditory stimulus signal,
i) repeat step h) until a reversal detection in accordance with the predetermined detection reversal criterion is fulfilled in the second path direction or until the two-dimensional boundary region is reached,
j) repeating steps e), f), g), h) and i) one or more times to transverse and record a stimuli path through the predetermined two-dimensional response space extending forth and back across the psychoacoustical threshold curve,
k) determining the psychoacoustical threshold curve based on at least a subset of the recorded parameter pairs indicating the stimuli path through the predetermined two-dimensional response space.

The audiological test apparatus or equipment may comprise a combination of standard audiological test devices, computing hardware and specifically tailored software-based test program(s) executed on a suitable programmable computing device such as a personal computer, laptop, tablet etc. forming part of the audiological test equipment. The standard audiological test devices or computing hardware may comprise the sound reproduction device, such as a calibrated loudspeaker, headphone, earphone etc., configured to apply the auditory stimulus signals to the hearing impaired patient and a calibrated sound processing unit (e.g. an audiometer). The audiological test apparatus may furthermore comprise a response detector configured to detect and record the listener's positive or negative detection responses to the presented auditory stimulus signals. The response detector preferably comprises a suitable interface to the listener to collect and record the responses as described in further detail below with reference to the appended drawings.

A fourth aspect of the invention relates to a computer readable data carrier comprising executable program instructions configured to cause the processor of the programmable computer to execute method steps a)-k) by execution of the test program. The computer readable data carrier may comprise various types of machine readable memory types such as semiconductor memories like flash memory, EEPROM, RAM, an optical disc and/or a magnetic disc etc. or any combination thereof.

### BRIEF DESCRIPTION OF THE DRAWINGS

Embodiments of the invention will be described in more detail below in connection with the appended drawings in which:

FIG. 1 is a schematic illustration of a method of determining a temporal masking curve (TMC) of a hearing impaired listener or patient in accordance with a first embodiment of the invention,

FIG. 2 is a schematic illustration of a method of determining a temporal masking curve (TMC) of a hearing impaired listener or patient in accordance with a second embodiment of the invention involving a modified detection reversal criterion,

FIG. 3 is a schematic illustration of a method of determining a temporal masking curve (TMC) of a hearing impaired listener or patient in accordance with a third embodiment of the invention,

FIG. 4 shows time domain characteristics of an auditory stimulus signal applied to the hearing impaired listener or patient during TMC testing,

FIG. 5A) shows frequency domain characteristics of an auditory stimulus signal applied to the hearing impaired listener or patient during a so-called notched-noise experiment in accordance with a fourth embodiment of the invention,

FIG. 5B) shows corresponding time domain characteristics of the auditory stimulus signal applied to the listener during the notched-noise experiment; and

FIG. 6 is a schematic illustration of a method of determining masked threshold curve of a hearing impaired listener or patient using a notched-noise stimulus signal in accordance with the fourth embodiment of the invention.

### DESCRIPTION OF PREFERRED EMBODIMENTS

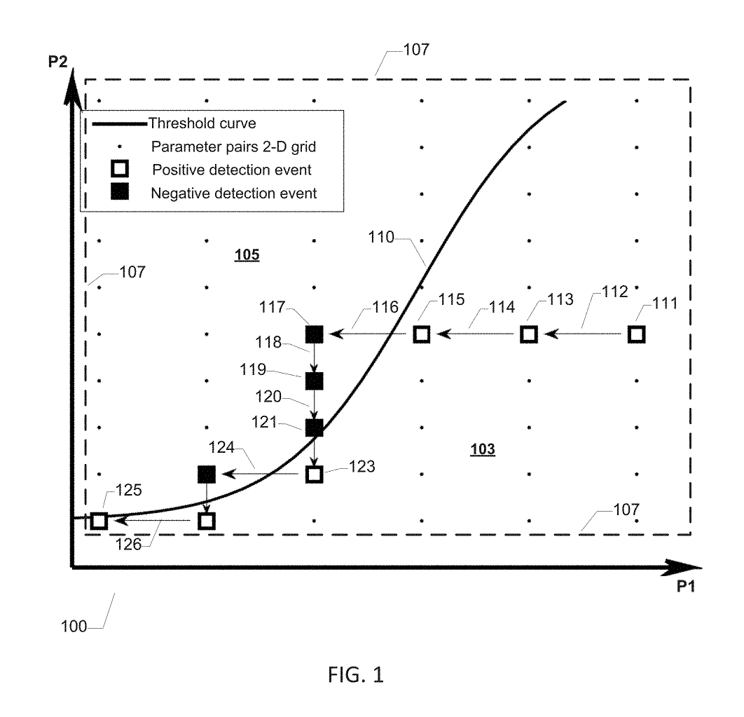

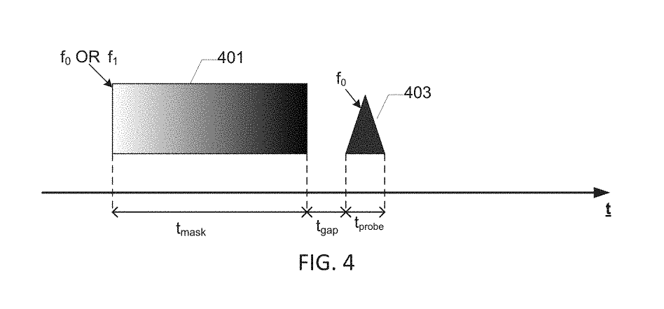

FIG. 1 is a schematic illustration of a method of determining a temporal masking curve (TMC) 110 of a hearing impaired listener, patient or test subject in accordance with a first embodiment of the invention. The skilled person will understand that the present methodology may be applied to the determination of numerous other types of psychoacoustical threshold curves. The temporal masking curve 110 is determined by selectively varying a first parameter and a second parameter of an auditory stimulus signal applied to the hearing impaired test person or patient. The first parameter P1 is a time gap between a masker tone burst and a probe tone burst of the auditory stimulus signal. P1 is mapped along the x-axis of graph 100. The second parameter P2 of the auditory stimulus signal is a variable sound pressure level of the masker tone. P2 is mapped along the y-axis of graph 100. The time domain characteristics of the auditory stimulus signal are schematically illustrated on FIG. 4. The auditory stimulus signal comprises the masker tone burst 401 of a predetermined duration tₘₐₛₖ followed by a time gap of variable duration t_{gap} and a probe tone or signal 403. The probe tone or signal 403 has a predetermined duration of t_{probe}. A frequency of the masker tone may be substantially identical to a frequency of the probe tone for performing a so-called on-frequency test. Both the probe tone and the masker tone are placed in the audible frequency range and preferably at a number of audiologically relevant frequencies between 100 Hz and 10 kHz for example at 1 kHz and 4 kHz. In one variant of the present methodology, the frequency of the masker tone differs from the frequency of the probe tone for performing a so-called off-frequency test of the listener's TMC. In the latter case, the frequency of the masker tone may be at least one-half octave lower than the frequency of the probe tone such as approximately one octave lower. The depicted temporal masking curve 110 is determined at any particular combination of masker tone frequency and probe tone frequency by fixing the level of the probe tone and finding such combinations of levels of the masker tone and positive values of the time gap (P2 mapped on the Y-axis) that mask the probe tone in 50% of the presentation cases (i.e. the 50% threshold). This is repeated for several, positive, values of the time gap t_{gap} (P1 mapped along the X-axis) inside a two-dimensional boundary region 107. The skilled person will understand that both the probe tone and the masker tone are placed in the audible frequency range and preferably at a test frequency of audiological relevance such as a test frequency between 100 Hz and 10 kHz for example at 500 Hz, 1 kHz or 4 kHz. The skilled person will understand that range of time gaps mapped along the x-axis tested may vary according to the specific nature of the temporal masking curve 110 and for example a priori determined hearing loss of the hearing impaired listener to be tested. The time gaps may be spaced linearly along the x-axis (e.g. 5, 10, 15, 20, 25, 30 . . . ms) or spaced exponentially (e.g. 5, 10, 20, 40, . . . ms) or even in a mixed way. In the present embodiment, the chosen time gap values may be 1, 2, 4, 8, 12, 16, 24, 32, 48, 64, 80, 96, 128, 160, 192, 224 and 256 ms although fewer parameter pairs are illustrated on the drawing for simplicity. The sound pressure level of the masker tone may be mapped in steps of a predetermined size along the y-axis for example step sizes between 2 and 6 dB.

The two-dimensional boundary region 107 may for example extend from a lower boundary value of P1 of 0 ms or 12 ms and an upper boundary value of P1 of 256 ms or less, for example about 100 ms. The lower boundary value of P2 (masker tone sound pressure level) as mapped along the y-axis of the two-dimensional boundary region 107 is preferably set in relation to the selected fixed level of the probe tone or probe signal. The probe tone level is in turn preferably fixed based on the listener's audiogram and must be clearly audible. In most cases the lower boundary level can well approximate the masker hearing threshold level for the time gap equaling 0 ms. However, 5 dB variations may occur and it may be preferable to set the lower or minimum boundary value of the masker sound pressure level to 7-10 dB below the chosen level of the probe tone. The upper bound on the sound pressure level of the masker tone may for example be chosen so as to avoid unnecessary discomfort of the listener. Consequently, depending on the actual hearing loss of the listener, the upper boundary limit or value may lie between 80 and 110 dB SPL.

The skilled person will appreciate that the hearing impaired listener's hearing threshold with or without the UCL level may have been determined via an ordinary audiogram measurement performed before the commencing the present methodology of determining the hearing impaired listener's temporal masking curve (TMC).

The audiological test apparatus or equipment utilized for the present method of determining the hearing impaired test person's temporal masking curve (TMC), and for determining the masked threshold curve in connection with the notched-noise experiment discussed below, may comprise a combination of standard audiological test devices/hardware and a specifically tailored software-based test program(s) executed on a suitable programmable computing device such as a personal computer, laptop, tablet etc. forming part of the audiological test equipment. The standard audiological test devices/hardware may comprise a sound reproduction device such as a calibrated loudspeaker, headphone or earphone configured to apply the auditory stimulus signals to the hearing impaired patient and a calibrated sound processing unit (e.g. an audiometer). The audiological test apparatus furthermore preferably comprises a response detector configured to detect and record the test person or listener's responses to the presented auditory stimulus signals. The response detector preferably comprises a suitable interface to the listener or test person to collect and record the responses, i.e. the listener's positive or negative detection of the relevant attribute/feature of the auditory stimulus signal.

The skilled person will appreciate that a major portion of the functionality of the audiological test apparatus may be built into the previously discussed personal computer, laptop, tablet etc. The test sequence, including the presentation of the auditory stimulus signals and the recording of the listener's responses may be controlled by a suitable test program executed on the personal computer, laptop, tablet etc. The test program may comprise a plurality of executable program instructions or code for example organized in various types of sub-routines, threads, sub-programs and APIs etc. The interface to the listener or test person may for example comprise a graphical user interface (GUI) presented on a touch-sensitive screen of the personal computer, laptop, tablet etc. The GUI may comprise various virtual buttons and/or input fields to detect and record the listener's responses. Likewise, the auditory stimulus signals may be generated by assistance of a soundcard and preexisting sound I/O ports of the personal computer, laptop, tablet etc.

Now reverting to the schematic illustration of the present methodology of determining the temporal masking curve 110 of the hearing impaired test person or listener depicted on graph 100 of FIG. 1, the method preferably begins with a determination of the placement of the two-dimensional boundary region 107 following the description above to determine upper and lower boundary limits or values. The two-dimensional boundary region 107 is preferably placed such that it surrounds an a priori estimated placement of the psychoacoustical threshold curve 110 to form a predetermined two-dimensional response space. In this manner, the two-dimensional boundary region 107 may define a restricted parameter space or area of the auditory stimulus signal to be searched to determine the psychoacoustical threshold curve 110. The use of the two-dimensional boundary region 107 is advantageous because it prevents the presentation of auditory stimulus signals with erroneous or superfluous parameter pairs to the listener as described below in further detail. The placement of the psychoacoustical threshold curve 110 can be estimated from the hearing loss of the listener in question and preexisting knowledge of the threshold curves of previously tested listeners with the same or corresponding hearing ability. Likewise, the overall shape of the psychoacoustical threshold curve 110 can for example be estimated from a priori knowledge of the hearing ability of humans. Thereby, it may be known at the start of the test procedure whether the sought after psychoacoustical threshold curve is monotonically increasing, as illustrated by the psychoacoustical threshold curve 110, or monotonically decreasing throughout the predetermined two-dimensional response space. In embodiments of the invention where the sought after psychoacoustical threshold curve has an a priori expected concave or convex shape, the present test procedure may be adapted by running separate test procedures for two separate sub-portions of the psychoacoustical threshold curve with different starting points and directions. In this manner, an increasing and a decreasing sub-portion of the concave or convex psychoacoustical threshold curve may be determined separately.

The predetermined two-dimensional response space comprises a positive response region 103 at a lower side of the estimated psychoacoustical threshold curve 110 and a negative response region 105 at an upper or second and opposite side of the estimated psychoacoustical threshold curve 110. A skilled person will understand that, depending on the experiment, a negative region may lie below the measured threshold curve and the positive region above the threshold curve. Auditory stimulus signals with parameter pairs placed in the positive response region 103 indicate that the particular attribute or feature of the auditory stimulus signal under investigation is audible to the hearing impaired listener. In the present embodiment where temporal masking curves are the type of psychoacoustical threshold curves to be determined, the audibility of the probe tone (403 of FIG. 4) is the investigated feature of the auditory stimulus signal. Conversely, auditory stimulus signals with respective parameter pairs placed in the negative response region 105 indicate that the particular attribute or feature of the presented auditory stimulus signal is inaudible to the hearing impaired listener. The skilled person will understand that the psychoacoustical threshold curve 110 may represent a 50% threshold or any other suitable threshold such as 25% or 75% depending on the experimental protocol. At the 50% threshold, the listener can detect the presence of the investigated feature of the auditory stimulus signal in 50% of the sound stimuli presentations.

Before commencing with the presentation of the auditory stimulus signals in connection with the present methodology of determining TMCs, the hearing impaired listener is instructed about the particular predetermined attribute/feature of the auditory stimulus signal that is to be detected—for example the presence or absence of the probe tone in the present TMC determination methodology. The listener instruction may comprise, or be followed by, a number of preliminary test runs or catch trials to accustom the listener to the detection task at hand. In the catch trials only the masker tone is played as discussed in further detail below.

The test procedure or testing methodology begins by determining a first parameter pair, schematically illustrated as open square 111 on the graph 100, comprising a first value of the time gap and a first value of masker tone sound pressure level where the first and second parameter values preferably are selected such that the corresponding auditory stimulus is situated well within the positive response region 103. Thereafter, a first auditory stimulus signal in accordance with this first parameter pair 111 is presented to the listener through a suitable sound reproduction device or devices such as a calibrated loudspeaker, headphone or earphone etc. The listener's positive or negative detection, i.e. audible or inaudible, of the probe tone of the first auditory stimulus signal is recorded for example in a suitable memory e.g. RAM, flash memory or magnetic disc memory of the previously discussed audiological test apparatus. Thereafter, a subsequent auditory stimulus signal(s) is presented to the listener in accordance with a subsequent parameter pair 113 arranged adjacent to the former, first, parameter pair 111 in a first path direction, as indicated by arrow 112, through the two-dimensional response space. The first path direction heads towards the a priori estimated placement of the psychoacoustical threshold curve 110.

The skilled person will appreciate that the step from the first parameter pair 111 to the subsequent parameter pair 113 is made between two neighboring parameter grid points along the first (horizontal) direction 112 of the two-dimensional response space. By stepping through the two-dimensional response space in the horizontal direction 112 the skilled person will understand that the value of only one parameter, the time gap P1, is altered between subsequent sound stimuli presentations while the second parameter which is the sound pressure level of the masker tone (P2) remains constant. This feature may be a significant advantage because listener responses are generally more consistent when only one property of the presented auditory stimulus signal changes at a time.

In the present embodiment, the two-dimensional response space comprises a predetermined two-dimensional parameter grid structure comprising a plurality of parameter pairs as indicated by the dots inside the boundary region 107. Each of the indicated parameter pairs comprises respective values of the time gap (P1) and masker tone sound pressure level (P2) and the two-dimensional response space within the boundary region 107 is only traversed by jumping or stepping between these parameter pairs of the dimensional parameter grid structure. However, the presence of the two-dimensional parameter grid structure is an entirely optional feature of the present embodiment and other embodiments may rely on an immediate computation of the value of any subsequent parameter pair once a preceding parameter pair has been evaluated for example to enable an adaptive approach to the selection of the step size and direction in the two-dimensional response space from a given parameter pair to any subsequent parameter pair. After evaluation of the subsequent auditory stimulus signal(s) associated with the subsequent parameter pair 113, the above auditory stimulus presentation and response recordation procedure is repeated, as schematically indicated by parameter pair 115 and stimulus path arrow 114 with selected parameter pair or pairs lying in the first horizontal direction of the two-dimensional grid structure such that the stimulus path crosses the psychoacoustical threshold curve 110 after a certain number of repetitions. As illustrated, the crossing of the psychoacoustical threshold curve 110 takes place at the presentation of the auditory stimulus signal associated with the parameter pair 117. This crossing of the psychoacoustical threshold curve 110 is reflected in a reversal of the listener's detection of the probe tone of the presented auditory stimulus signal, i.e. in the present situation from an “audible/Yes” response to an “inaudible/No” response. An “inaudible/No” response by the listener is indicated by a black filled-out square in the graph 100. Hence, an initial response reversal takes place along the first horizontal direction of the two-dimensional grid structure when stepping from the parameter pair 115 to the parameter pair 117. In this manner, the approximate placement of the threshold curve in terms of time gap (P1 value) and corresponding sound pressure level of the masker tone (P2 value) has been determined. Once, the listener's response reversal is verified to a satisfactory degree, the direction to the subsequent parameter pair of the auditory stimulus signal is changed to a second path direction which is different from the first path direction and its reverse, i.e. the horizontal direction along path arrows 112, 114 in the illustrated example. The second path direction also heads towards the a priori estimated placement of the psychoacoustical threshold curve 110 to ensure rapid stepping toward the curve 110. The second path direction is preferably substantially perpendicular to the horizontal direction as indicated by vertical path direction arrows 118, 120.

This leads to the previously discussed benefits with varying one parameter only between subsequent auditory stimulus presentations when traversing the predetermined two-dimensional response space. In this case, the masker tone sound pressure level (P2 value) only is varied when moving in the vertical direction of the two-dimensional grid structure and the time gap (P1) only is varied when moving in the horizontal direction of the two-dimensional grid structure. The skilled person will appreciate that the change from a first path direction, e.g. the illustrated horizontal direction, to a second path direction, e.g. the illustrated vertical direction, which is different from the first path direction and its reverse, has the important methodological advantage that most of the experimental time is spent in the vicinity of the sought after psychoacoustical threshold curve. Hence, by avoiding the immediate return path leading back across the psychoacoustical threshold curve 110 in the already tested horizontal direction (against path direction arrows 116, 114, 112), the number of presented auditory stimulus signals to estimate the threshold is minimized. This feature has the benefit that the length or duration of entire test procedure for determining a given type of psychoacoustical threshold curve 110 is markedly reduced compared to prior art methodologies wherein auditory stimulus signals proximate to the estimated threshold region are repeatedly presented. Another way to look at this feature of the present methodology of determining psychoacoustical threshold curves is that the methodology increases the number of response reversals between the Yes region 103 and the No region 105.

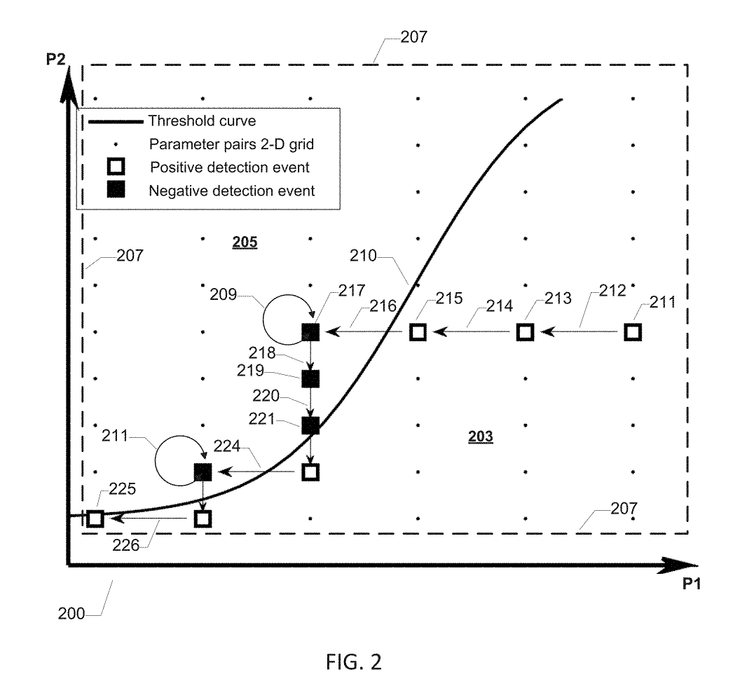

The skilled person will appreciate that a response reversal may be accepted immediately after the listener's first response reversal taking place when stepping from parameter pair 115 to parameter pair 117 or that more elaborate criteria may be applied to obtain further confidence in the validity of the initial or first response reversal before changing the path direction to the second path direction. Hence, the present methodology comprises a predetermined detection reversal criterion which must be fulfilled to accept the validity of a given response reversal. A very simple detection reversal criterion is schematically illustrated on FIG. 1 where the initial response reversal between parameter pairs 115 and 117 immediately is accepted such that the subsequently selected auditory stimulus signal has parameter pair 119. As illustrated, the parameter pair lies on the two-dimensional grid structure along the new, vertical, direction. Alternative forms of the predetermined detection reversal criterion are discussed below in connection with FIGS. 2 & 3. The skilled person will understand that more elaborate versions of the predetermined detection reversal criterion may lead to higher confidence in any particular reversal detection at the expense of an increasing number of stimuli presentations and experimental time.

Once, the listener's response has fulfilled, or complied with, the predetermined detection reversal criterion, the presentation of one or more subsequent auditory stimulus signal(s) proceed along the vertical or second path direction as indicated by path direction arrow s 118, 120 as discussed above. The one or more subsequent auditory stimulus signal(s) may for example comprise stimulus signals according to parameter pairs 119 and 121 which both lead to negative detections of the probe tone, i.e. inaudible. The presentation of the subsequent auditory stimulus signals accordingly proceeds until parameter pair 123 is presented to the listener. The listener detects the probe tone of the auditory stimulus signals according to parameter pair 123 thereby leading to a second response reversal finding at the parameter pair 123 as illustrated by the depicted rectangular open box symbol. The values of the first and second parameters of the parameter pair 123 are recorded by the test program. Thereafter, the path or step direction through the predetermined two-dimensional response space reverts to the horizontal direction, as indicated by path direction arrow 124 pointing towards the a priori estimated placement of the psychoacoustical threshold curve 110, and the above-outlined test steps are repeated a certain number of times for example until the parameter pair 125, situated close to a lower left corner of the two-dimensional boundary region 107, is reached. Thereby, a stimuli path through the predetermined two-dimensional response space is traversed following the path direction arrows 112, 114, 116, 118, 120, 122, 124, 126 etc. This stimuli path is extending forth and back across the psychoacoustical threshold curve 110 and comprises a very small number of individual auditory stimulus signal presentations.

If one of the four boundary limits of the two-dimensional boundary region 107 is reached during the test procedure, a corrective action is preferably taken because this incident may indicate an erroneous listener response for example caused by fatigue or lacking understanding of the detection task at hand. Depending on which corner or which boundary limit of the two-dimensional boundary region 107 that is reached, the following cases exist:

1. Hitting upper-left corner: This should be deep inside the negative response region 105 and hence a Yes/audible response is highly improbable. If that nevertheless happens, the listener should be examined for understanding of the task at hand. Otherwise, when a No response is achieved, two scenarios can happen. If a current path direction was upwards in the two-dimensional response space 107, the masker level is kept and the time gap is increased to simplify the task. Thereby, the positive or Yes response region 103 should be reached—i.e. the path direction is away from the corner and rightwards. Alternatively, if the current direction was leftwards the downwards direction is assumed.
2. Lower-Right Corner: This is a directly opposite case to upper-left corner and a Yes/audible listener response is expected and the procedure continues with a (leftward or upward) movement towards the negative response region 105.
3. Lower-left corner: This corner will be reached as a consequence of following the general left-down direction through the response space. In such a case the general direction is preferably switched into right-up—i.e. the procedure bounces back and the psychoacoustical threshold curve 110 curve is re-sampled.
4. Upper-right corner: This corner will be reached as a consequence of following the general right-up direction. In such a case the general direction is switched into left-down—i.e. the procedure bounces back and the psychoacoustical threshold curve 110 is re-sampled.
5. In case of hitting one of the four boundary limits, delimiting the two-dimensional boundary region 107, outside the four corners, the path direction through the boundary region is altered such that the simplification or complication of the detection task at hand is continued. For instance, if the left vertical boundary limit is reached or hit, this means that (when response region 105 is a negative detection region above the threshold curve) the complexity of the detection task was being increased, but the predetermined detection reversal criterion has not yet been fulfilled or complied with. Hence, upwards direction is assumed and the general path direction through the two-dimensional response space is changed to right and up. If the upper boundary limit is reached that means, again, that the complexity was being increased, but the reversal criterion indicating reaching the NO region was not met yet. In response to this situation, the path direction is changed to leftwards and the left-down general direction is assumed. The skilled person will understand that, by analogy, reaching or hitting the right and lower boundary limits corresponds to simplification of the detection task and new downward and rightward step directions are initiated, respectively.

In some embodiments of the invention, hitting any of the boundary limits of the two-dimensional boundary region 107 may be used as a stopping criterion. In response to meeting the stopping criterion, the presentation of auditory stimulus signals recording or collection of listener's responses ceases. The psychoacoustical threshold curve in question is estimated from the recorded collection of parameter pairs with associated positive or negative detections of the listeners. In one such embodiment, hitting the upper boundary limit is used as a stopping criterion. Alternative or additional stopping criteria may be applied for example: reaching a predefined maximum number of listener detection responses or reaching a predefined number of changes between the first and second path direction through the two-dimensional response space.

The skilled person will understand that the described behavior of the test methodology outlined under points 1-5 above at the boundary limits and the corners of the two-dimensional boundary region 107 may apply to the case when a monotonically increasing threshold curve is being investigated such that the negative response region is located above the threshold curve. The preferred behavior at the boundary limits in case of other threshold curve shapes (e.g. monotonically decreasing) can easily be derived by following the outlined rule of continuing the simplification or complication of the detection task.

Finally, the psychoacoustical threshold curve 110 is determined based on the recorded parameter pairs indicating the above-mentioned stimuli path through the two-dimensional response space. There are several ways to estimate the psychoacoustical threshold curve 110 from the recorded or stored parameter pairs 111, 113, 115, 117, 119, 121, 123, 125 held in the memory of the audiological test equipment. The stored parameter pairs in the audiological test equipment can be viewed as a set of vectors. These vectors contain coordinates (in this embodiment, the time gap value and the sound pressure level of the masker tone or masker level) and the listener's responses to the auditory stimuli characterized by these coordinates. A first step to determine the psychoacoustical threshold curve 110 is creating a map of listener responses. The map may have the same format as illustrated on FIG. 1 which has been used during the stimuli presentation procedure. For each unique gap-masker level pair used in the methodology a mean yes-rate is estimated which equals the number of Yes/audible responses over the total number of questions asked at this specific parameter pair. In this manner a first approximation of the psychoacoustical threshold curve 110 is obtained. Next, focus is put on all the parameter pairs that possess the same time gap values. The latter set of points may be designated “a column”. Using linear interpolation coordinates of parameter pairs are found that satisfy a necessary condition—i.e. correspond to 50% yes rate. Subsequently, a Heaviside step function or unit step function is found that minimizes the fitting error within such a single column of parameter pairs that possess the same time gap values. Being a minimizer of the fitting error is understood here as fulfilling a sufficient condition of being an estimate of the 50% point on a two-dimensional function. Formally, a one-parameter one-dimensional psychometric function is fitted to the data set forming a single column. The masker level that corresponds to the position of the step may be considered an estimate of the 50% point on the two-dimensional psychometric function. There may exist more than one Heaviside step function with same minimum fitting error, but different positions of the step (masker levels). The mean of such multiple masker levels may reasonably serve as a final estimate of the 50% threshold of the masker level for the specific time gap. This single-column fitting procedure is applied to all columns/time gap coordinates such that a set of 50% points on the two-dimensional psychometric function is found. A fitting procedure which is similar to the above-described column wise fitting can be executed for rows of the two-dimensional response space, i.e. keeping the masker level constant and finding the 50% time gap threshold.

FIG. 2 is a schematic illustration of a method of determining a temporal masking curve (TMC) 210 of a hearing impaired listener, patient or test subject in accordance with a second embodiment of the invention. Corresponding features of the first embodiment of the invention and the present embodiment have been provided with corresponding reference numerals to ease comparison. The present methodology applies a modified detection reversal criterion compared to the first embodiment. The present test procedure or testing methodology is otherwise identical to the one discussed in detail above in connection with the first embodiment, The testing methodology begins by determining a first parameter pair, schematically illustrated as open square 211 on the graph 200 comprising a first value of the time gap and a first value of masker tone sound pressure level. As before the first parameter pair is preferably selected such that the corresponding auditory stimulus is situated well within the positive response region 203. Thereafter, the test procedure proceeds as described above following path direction arrows 212, 214, 216 and parameter pairs 213, 215, 217. However, when the listener's initial response reversal is detected between parameter pairs 215 and 217 this is not immediately accepted such that a change of direction of the stimuli path through the two-dimensional response space is carried out. Instead, the auditory stimulus signal with parameter pair 217 is presented again to the listener as schematically illustrated by repetition arrow 209. If the listener then confirms his/hers initial response reversal, then the change of direction of the stimuli path is effected and the subsequently selected auditory stimulus signal has parameter pair 219 positioned along the new, vertical, direction of the grid structure following path direction arrow 218. The repletion of the presentation of the stimulus signal with parameter pair 217 may provide a higher confidence in the listener's response, i.e. a lacking detection of the probe tone for the present parameter pair. On the other hand, if the listener fails to confirm his/hers initial response reversal at the repeated presentation of the auditory stimulus signal with parameter pair 217, several options are available. In one embodiment, a subsequent parameter pair, which is arranged in the same direction as the former parameter pair is selected, i.e. following the horizontal direction as indicated by arrows 212, 214, 216. Thereafter the corresponding auditory stimulus signal is presented to the listener. This additional horizontal step will bring the auditory stimulus signal further into the negative response region 205 and increase the likelihood of the probe tone being judged inaudible by the listener such that a negative detection event is reached again. Once again, the confidence in the listener's response can be increased by presenting the auditory stimulus one more time to receive the expected negative detection result. Only after having received at least two consecutive negative detection responses, the change of direction of the stimuli path may now be carried out due to the increased confidence in having entered the negative response region 205. Therefore, the parameter pair of the subsequent auditory stimulus signal may be selected along the vertical direction of the two-dimensional grid structure. As before, the parameter pair of the subsequent auditory stimulus signal is selected such that the direction to the parameter pair of the subsequent auditory stimulus heads towards the a priori estimated placement of the temporal masking curve 210 as illustrated by path direction arrow 218.

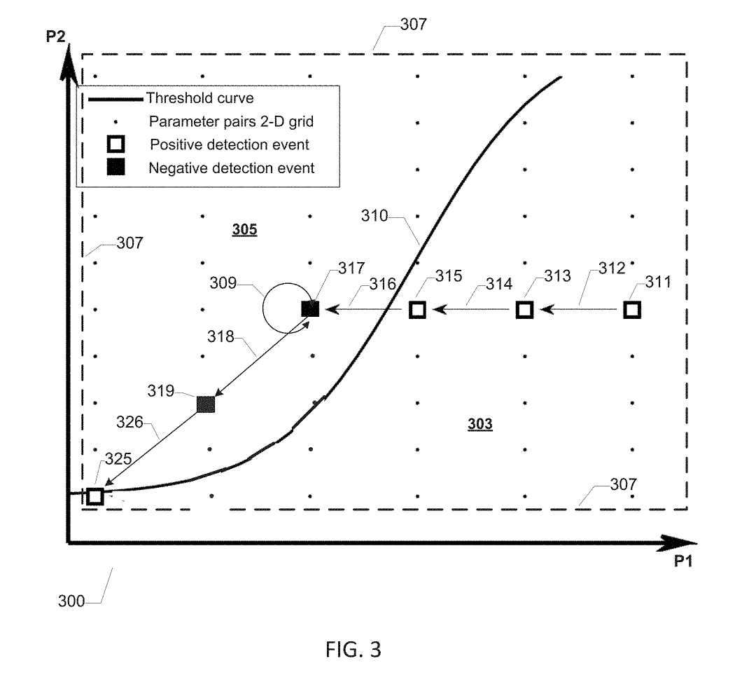

FIG. 3 is a schematic illustration of a method of determining a temporal masking curve (TMC) 310 of a hearing impaired listener, patient or test subject in accordance with a third embodiment of the invention. Corresponding features of the first embodiment of the invention and the present embodiment have been provided with corresponding reference numerals to ease comparison. The present methodology applies the same modified detection reversal criterion as utilized in the second embodiment of the invention but may alternatively use the same detection reversal detection criterion as the first embodiment. The present test procedure or testing methodology differs from the one discussed in detail above in connection with the first and second embodiments of the invention by stepping through the two-dimensional response space in first and second path directions which are not perpendicular. In the present embodiment, the selected parameter pairs may be placed on the two-dimensional grid structure (schematically indicated by the regular dots) or placed outside the two-dimensional grid structure. As mentioned previously, the two-dimensional grid structure may be entirely absent as discussed before. The testing methodology begins by determining a first parameter pair, schematically illustrated as open square 311 on graph 300 comprising a first value of the time gap and a first value of masker tone sound pressure level. As before the first parameter pair is preferably selected such that the corresponding auditory stimulus is situated well within the positive response region 303 of the two-dimensional boundary region 307 defining the two-dimensional response space in the present embodiment. Thereafter, the test procedure proceeds as described above by stepping through the two-dimensional response space in horizontal direction following path direction arrows 312, 314, 316 and parameter pairs 313, 315, 317. When the listener's initial response reversal is detected between parameter pairs 315 and 317 this response reversal is retested as schematically illustrated by repetition arrow 309 in a manner similar to the one discussed in connection with the second embodiment of the invention. When the detection reversal detection criterion has been fulfilled at parameter pair 317, a change of direction of the stimuli path from the first, horizontal, direction to a second path direction, as indicated by direction arrow 318 is effected. The second path direction is different from the first, horizontal, direction and its reverse (i.e. against the path direction arrows 316, 314) and heads towards the expected position of the temporal masking curve 310. In practice, this means that both parameter values of the auditory stimulus signal are changed when stepping from parameter pair 317 to the subsequent parameter pair 319. The next step through the stimuli path proceeds in the second path direction to the subsequent parameter pair 325. This may be the endpoint of the test procedure.

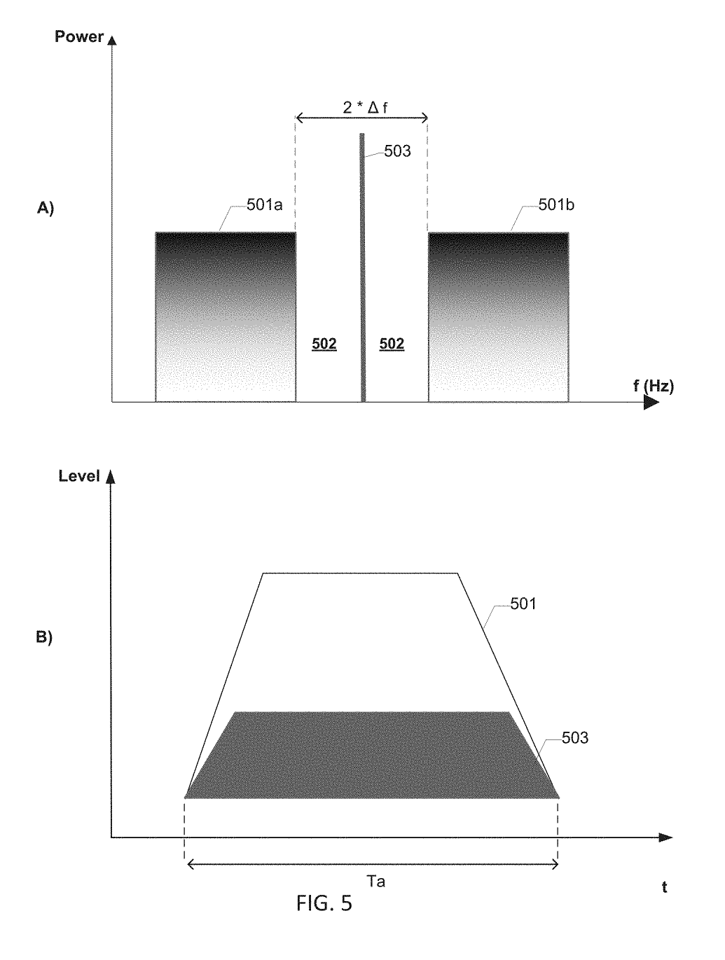

FIG. 5A) shows frequency domain characteristics of an auditory stimulus signal applied to the hearing impaired listener or patient during a so-called notched-noise experiment. FIG. 5B) shows schematically corresponding time domain characteristics of the auditory stimulus signal applied to the listener during the notched-noise experiment. The purpose of the notched-noise methodology or experiment is to collect data that can be used to estimate properties of the auditory filters (attributed to cochlear action) of the hearing impaired listener or patient. The skilled person will understand that the notched-noise methodology or experiment may be used to determine auditory filter shapes of normal hearing subjects as well. Properties of interest are overall shape of the auditory filters and in particular, a bandwidth of each of the auditory filters. The bandwidth of the auditory filter indicates a spectral resolution of the hearing impaired or normal hearing subject's auditory system. Hearing-impaired listeners tend to have lower spectral resolution (larger bandwidths) of the auditory filters.

The notched-noise experiment is a simultaneous masking experiment, which means that the presented auditory stimulus signal comprises a masking stimulus 501 (shortly “the masker”) and the stimulus 503 that is being masked (“probe tone”). The masker 501 and the probe tone 503 are preferably presented to the listener at the same time as indicated on FIG. 5B). The listener's task during the notched-noise experiment is to detect and report the presence or absence of the probe tone 503 in the presented auditory stimulus signal. Hence, the predetermined attribute/feature of the presented auditory stimulus signal is the audibility of the probe tone 503. The probe tone 503 may be a sinusoidal tone with fixed frequency. The masker 501 preferably comprises of two spectrally separated frequency bands of noise 501a, 501b. The separate frequency bands of noise 501a, 501b may be symmetrical as illustrated in the present embodiment. Alternatively, asymmetrical noise frequency bands 501a, 501b may be utilized as masker to reveal the asymmetry of the auditory filter shape. In both cases, the probe tone 503 is positioned in the frequency domain in such a way that the frequency of the probe tone falls between the two spectrally separated frequency bands of noise 501a, 501b. In other words, the two spectrally separated frequency bands of noise 501a, 501b are separated by notch band 502 with a notch bandwidth of 2*Δf and the probe tone is situated in that notch band 502. These characteristics of the auditory stimulus signal are selected because the listener typically has the best performance if he/she focuses on the output of the auditory filter which possesses a centre frequency closest to the frequency of the presented probe tone. The case where the listener focuses on the output of a different auditory filter is called the “off-frequency” listening. The time-domain representations of the masker 501 and the probe tone 503 on FIG. 5B) are schematically illustrated as comprising respective trapezoid envelopes or outlines. The trapezoidal envelope symbolizes that the masker 501 and the probe tone 503 each may possesses a roll-on and roll-off signal segments. These signal segments may limit the spectral splatter that would occur in case of abrupt on-set and termination of a probe tone or noise signal. An exemplary length of such a roll-on and roll-off signal segment is between 2 ms and 20 ms such as between 5 and 20 ms. The roll-on and roll-off signal segments may comprise half a Hann window. The overall duration of the auditory stimulus signal Ta may lie between 30 ms and 500 ms such as between 100 and 500 ms for example 200 ms.

During prior art or traditional notched-noise experiments, a noise spectral density is kept fixed and during each individual test procedure and the bandwidth of 2*Δf of the notch band 502 is kept fixed. The level of the probe tone is the only varying parameter during the test procedure. After the threshold of the probe tone is estimated, a new bandwidth of 2*Δf of the notch band 502 is selected and the experiment is repeated with the new settings to estimate the next signal threshold.

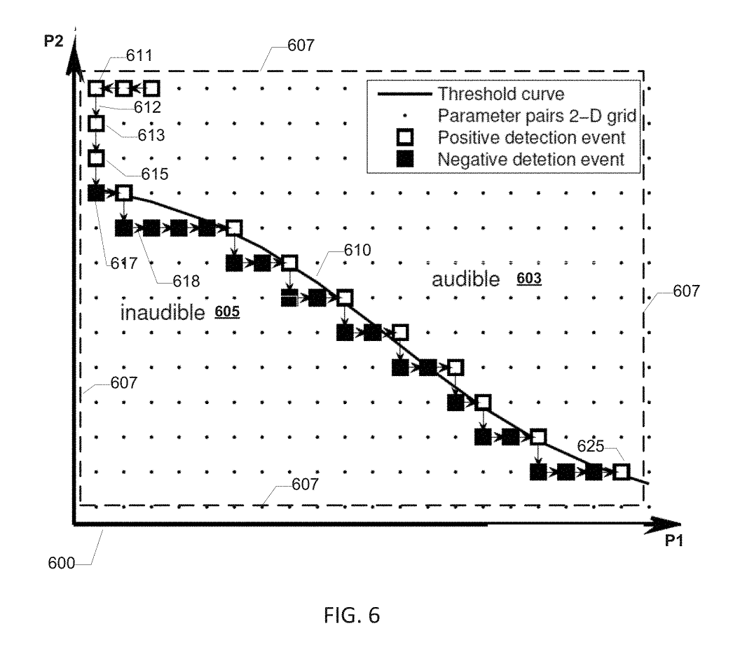

In contrast, the notch bandwidth (2*Δf) of the notch band 502 and the level of the probe tone 503 are varied selectively during the test procedure using the present methodology of the determining the masking curve using the notched-noise experiment. Consequently, notch bandwidth 2*Δf of the notch band 502 is a first parameter P1 of the auditory stimulus signal and the sound pressure level of the probe tone 503 is second parameter P2 of the auditory stimulus signal. P2 is mapped along the y-axis of graph 600 of FIG. 6 while P1 is mapped along the x-axis. As mentioned above, FIG. 6 is a schematic illustration of the application of present methodology to determining the masking curve 610 under the notched-noise test procedure. This masking curve 610 is a psychoacoustical threshold curve which may be used to derive characteristics of the hearing impaired listener's or test subject's auditory filters, in particular a bandwidth of one or more auditory filters.

The depicted masking curve 610 may be obtained by finding such combinations of the notch bandwidth 2*Δf and level of the probe tone 503 that mask the probe tone 503 in 50% of the presentation cases (i.e. the 50% threshold). The skilled person will understand that both the probe tone and the noise bands 501a, 501b are placed in the audible frequency range. The probe tone preferably has a frequency of audiological relevance such as a frequency between 100 Hz and 10 kHz for example at 500 Hz, 1 kHz or 4 kHz. The skilled person will understand that the range of notch bandwidths (2*Δf) mapped along the x-axis may vary according to the specific nature of the masking curve 610 for example a priori determined hearing loss of the hearing impaired listener to be tested. The sound pressure level of the probe tone 503 is mapped along the y-axis and may use grid steps of a predetermined size for example step sizes between 2 and 6 dB. The present determination of the masking curve 610 is carried out within a predetermined two-dimensional response space comprising a positive response region 603 and a negative response region 605 placed on opposing sides of the masking curve 610. The predetermined two-dimensional response space preferably comprises a two-dimensional boundary region 607 with predetermined lower and upper boundary limits at both of the orthogonal P1 and P2 directions to prevent presentation of auditory stimulus signals with erroneous or superfluous parameter for the reasons discussed in detail above with reference to the previous embodiments of the present methodology.

As discussed previously, the placement of the masking curve 610 within the predetermined two-dimensional response space can be estimated from the hearing loss of the listener in question and preexisting knowledge of the threshold curves of previously tested listeners with the same or corresponding hearing ability. Likewise, the overall shape of the psychoacoustical threshold curve 610 can for example be estimated from a priori knowledge of the hearing ability of normal hearing or hearing impaired individuals as the case may be. Thereby, it may be known at the start of the test procedure whether the sought after psychoacoustical threshold curve is monotonically decreasing, as illustrated by the threshold curve 610, or monotonically increasing throughout the predetermined two-dimensional response space.

Before commencing with the presentation of the auditory stimulus signals in connection with the present notched-noise experiment or methodology, the hearing impaired listener is preferably instructed about the particular predetermined attribute/feature of the auditory stimulus signal to be detected—for example the presence or absence of the probe tone 503 in the presented the auditory stimulus signal. The listener instruction may comprise, or be followed by, a number of preliminary test runs or catch trials to accustom the listener to the detection task at hand as discussed before.

The test procedure or testing methodology begins by selecting a first parameter pair, schematically illustrated as open square 611 on the graph 600, comprising a first value of the notch bandwidth 2*Δf and a first value of probe tone sound pressure level where the first and second parameter values preferably are selected such that the corresponding auditory stimulus signal is situated well within the positive response region 603. Thereafter, a first auditory stimulus signal in accordance with this first parameter pair 611 is presented to the listener through a suitable sound reproduction device or devices such as a calibrated loudspeaker, headphone or earphone etc. The listener's positive or negative detection, i.e. audible or inaudible, of the probe tone of the first auditory stimulus signal is recorded as described before and a subsequent auditory stimulus signal(s) is presented to the listener in accordance with a subsequent parameter pair 613 arranged adjacent to the former, first, parameter pair 611 in a first path direction, as indicated by arrow 612, through the two-dimensional response space. As previously discussed, the first path direction heads towards the a priori estimated placement of the masking curve 610. The skilled person will appreciate that the step from the first parameter pair 611 to the subsequent parameter pair 613 is made between two neighboring parameter grid points along the first (vertical) direction 612 of the two-dimensional response space. By stepping through the two-dimensional response space in the vertical direction 612 the skilled person will understand that the value of only one parameter P2, the sound pressure level of the probe tone 503 is altered between subsequent stimuli presentations while the second parameter, P1 which is the notch bandwidth 2*Δf, of the auditory stimulus signal, remains essentially constant. This feature may be a significant advantage because listener responses are generally expected to be more consistent when only one property of a presented auditory stimulus signal changes at a time.

In the present embodiment, the two-dimensional response space comprises a predetermined two-dimensional parameter grid structure comprising a plurality of parameter pairs as indicated by the dots inside the boundary region 607. Each of the indicated parameter pairs comprises respective values of the notch bandwidth 2*Δf (P1) and probe tone sound pressure level (P2). The two-dimensional response space within the boundary region 607 is only traversed by jumping or stepping between these parameter pairs of the dimensional parameter grid structure. However, the presence of the two-dimensional parameter grid structure is an entirely optional feature of the present embodiment and other embodiments may rely on an immediate computation of the value of any subsequent parameter pair once a preceding parameter pair has been evaluated using an adaptive approach as discussed above.

After evaluation of the subsequent auditory stimulus signal(s) associated with the subsequent parameter pair 613, the above auditory stimulus presentation and response recordation procedure is repeated in the manner discussed above in connection with e.g. the first embodiment of the methodology. As illustrated, the first crossing of the masking curve 610 takes place at the presentation of the auditory stimulus signal associated with the parameter pair 617. This first crossing of the masking curve 610 is reflected in a reversal of the listener's detection of the probe tone of the presented auditory stimulus signal, i.e. in the present situation from an “audible/Yes” (positive) detection event to an “inaudible/No” (negative) detection event. As previously discussed, the “inaudible/No” response by the listener is indicated by a black filled-out square in the graph 600. Hence, the first response reversal takes place along the first vertical direction of the two-dimensional grid structure when stepping from the parameter pair 615 to the parameter pair 617 on the grid structure. In this manner, the approximate placement of the threshold curve in terms of notch bandwidth (P1 value) and corresponding sound pressure level of the probe tone (P2 value) has been determined. Once, the listener's response reversal is verified to a satisfactory degree, the direction to the subsequent parameter pair of the auditory stimulus signal is changed to a second path direction along path arrow 612 in the illustrated example. The second path direction indicated by path arrow 618 also heads towards the a priori estimated placement of the masking curve 610 to ensure rapid stepping toward the curve 610. The second path direction is preferably substantially perpendicular to the vertical, or first, direction as indicated by horizontal direction arrow 618. The predetermined two-dimensional response space within the two-dimensional boundary region 607 is subsequently traversed in a corresponding manner to the previously discussed first embodiment of the methodology. In the present case, the level of the probe tone (P2 value) only is varied when moving along the vertical direction of the two-dimensional grid structure and the notch bandwidth (P1 value) only is varied when moving along the horizontal direction of the two-dimensional grid structure. The skilled person will appreciate that the change from a first path direction, e.g. the illustrated horizontal direction, to a second path direction, e.g. the illustrated vertical direction, which is different from the first path direction and its reverse, possesses the same methodological advantages as discussed before.

The skilled person will appreciate that a response reversal may be accepted immediately after the listener's first response reversal taking place when stepping from parameter pair 615 to parameter pair 617 or that more elaborate criteria may be applied using the criteria options discussed before to obtain further confidence in the validity of the initial or first response reversal. Overall, a stimuli path through the predetermined two-dimensional response space is traversed extending forth and back across the masking curve 610 and comprises a very small number of individual auditory stimulus signal presentations. The above-outlined test steps are repeated a certain number of times for example until a particular predetermined parameter pair, for example pair 625 situated close to a lower right corner of the two-dimensional boundary region 607, is reached to indicate that the relevant or desired portion of the masking curve 610 has been traversed. If one of the four boundary limits of the two-dimensional boundary region 607 is reached during the test procedure, the previously discussed corrective actions may be carried out.

1. A method of determining a psychoacoustical threshold curve by selectively varying a first parameter and a second parameter of an auditory stimulus signal applied to a test subject/listener, comprising steps of:
a) determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve,
b) instructing the listener to detect a predetermined attribute/feature of the auditory stimulus signal,
c) determining a first parameter pair comprising a value of the first parameter and a value of the second parameter where the first parameter pair is situated in the positive response region,
d) presenting a first auditory stimulus signal in accordance with the first parameter pair to the listener through a sound reproduction device and recording the listener's positive or negative detection of the predetermined attribute/feature of the first auditory stimulus signal,
e) presenting a subsequent auditory stimulus signal(s) to the listener in accordance with a subsequent parameter pair following a former parameter pair in a first path direction through the two dimensional response space; wherein the first path direction heads towards the a priori estimated placement of the psychoacoustical threshold curve,
f) recording the listener's positive or negative detection of the predetermined attribute/feature of the subsequent auditory stimulus signal(s) and repeat steps e) and f) until a reversal detection in accordance with a predetermined detection reversal criterion is fulfilled in the first path direction or until the two-dimensional boundary region is reached,
g) select a subsequent parameter pair following the former parameter pair in a second path direction, differing from the first path direction and its reverse, in the predetermined two-dimensional response space, wherein the second path direction heads towards the a priori estimated placement of the psychoacoustical threshold curve,
h) presenting a subsequent auditory stimulus signal in accordance with the subsequent parameter pair to the listener and recording the listener's positive or negative detection of the predetermined attribute/feature of the subsequent auditory stimulus signal,
i) repeat step h) until a reversal detection in accordance with the predetermined detection reversal criterion is fulfilled in the second path direction or until the two-dimensional boundary region is reached,
j) repeating steps e), f), g), h) and i) one or more times to traverse and record a stimuli path through the predetermined two-dimensional response space extending forth and back across the psychoacoustical threshold curve,
k) determining the psychoacoustical threshold curve based on at least a subset of the recorded parameter pairs indicating the stimuli path through the predetermined two-dimensional response space.

2. A method of determining a psychoacoustical threshold curve according to claim 1, wherein the second path direction extends substantially orthogonally to the first path direction in the predetermined two-dimensional response space.

3. A method of determining a psychoacoustical threshold curve according to claim 1, wherein the predetermined two-dimensional response space comprises a predetermined two-dimensional parameter grid structure comprising a plurality of parameter pairs comprising respective values of the first and second parameters.

4. A method of determining a psychoacoustical threshold curve according to claim 3, wherein the subsequent parameter pair of each of steps e) and g) is placed adjacent to the former parameter pair in the two dimensional parameter grid structure.

5. A method of determining a psychoacoustical threshold curve according to claim 1, wherein the predetermined detection reversal criterion comprises:
identifying an initial response reversal in the first path direction or an initial response reversal in the second path direction,
selecting the subsequent parameter pair in opposite direction of the former parameter pair, and
present the subsequent auditory stimulus signal in accordance with the subsequent parameter pair.

6. A method of determining a psychoacoustical threshold curve according to claim 1, wherein the predetermined detection reversal criterion comprises:
identifying an initial detection reversal in the first path direction or an initial detection reversal in the second path direction,
repeating the presentation of the auditory stimulus signal that led to the initial detection reversal,
if the reversal detection is confirmed then proceed to step e) or step g) to proceed in an opposite path direction to a current direction; or
if the reversal detection is denied then determine a subsequent parameter pair arranged in the same path direction as the former parameter pair, and
present a subsequent auditory stimulus signal in accordance with the subsequent parameter pair.

7. A method of determining a psychoacoustical threshold curve according to claim 1, wherein the psychoacoustical threshold curve is either monotonically decreasing throughout the predetermined two-dimensional response space or monotonically increasing throughout the predetermined two-dimensional response space.

8. A method of determining a psychoacoustical threshold curve according to claim 1, wherein each of the auditory stimulus signals comprises a masker tone and a probe/signal tone separated by a time gap; and
the predetermined attribute/feature of each of the auditory stimulus signals being the probe/signal tone; and
wherein the first parameter of the auditory stimulus signals is associated with a signal property of the masker tone and the second parameter is associated with either a signal property of the probe tone or a property of the time gap.

9. A method of determining a psychoacoustical threshold curve according to claim 8, wherein the first parameter of the auditory stimulus signals is a level of the masker tone and the second parameter of the auditory stimulus signals is the time gap between the masker tone and the probe tone such that the psychoacoustical threshold curve represents a temporal masking curve (TMC) of the test subject/listener.

10. A method of determining a psychoacoustical threshold curve according to claim 9, wherein time gap values are mapped along a first axis of the two-dimensional response space and levels of the masker tone are mapped along a second axis, orthogonal to the first axis, of the two-dimensional response space.

11. A method of determining a psychoacoustical threshold curve according to claim 10, wherein the parameter pairs mapped to the two-dimensional response space at least comprises:
time gap values between 1 ms and 200 ms with a predetermined linear or logarithmic gap spacing; and
masker level values between 10 dB SPL and 85 dB SPL with a predetermined linear or logarithmic level spacing.

12. A method of determining a psychoacoustical threshold curve according to claim 1, comprising a measurement of an audiogram of the listener prior to performing step a) of claim 1.

13. A method of determining a psychoacoustical threshold curve according to claim 9, wherein a lower bound of the two-dimensional response space is determined from a level (fixed) of the probe tone and an upper bound is determined based on a hearing loss of the listener at the frequency of the masker signal.

14. A method of determining a basilar membrane input/output curve of a listener at one or several audiologically relevant test frequencies based on temporal masking curves, comprising steps of:
a) selecting a first test frequency,
b) applying the method of determining the temporal masking curve according to claim 9 to the listener a first time where a frequency of the probe tone is equal to the first test frequency and set a frequency of the masker tone at least one-half octave lower than the first test frequency,
c) record and store in a data memory device a first temporal masking curve resulting from the first time of application of the method of determining the temporal masking curve,
d) applying the method of determining the temporal masking curve according to claim 9 to the listener a second time where the frequency of the probe tone and the frequency of the masker tone are both substantially equal to the first test frequency,
e) record and store in the data memory device a second temporal masking curve resulting from the second time of application of the method of determining the temporal masking curve,
f) compute the listener's basilar membrane input/output curve at the first test frequency based on the first and second temporal masking curves.

15. An audiological test apparatus for determining a psychoacoustical threshold curve by selectively varying a first parameter and a second parameter of an auditory stimulus signal applied to a test subject or listener, the apparatus comprising:
a programmable computer controlled by a test program comprising a plurality of executable program instructions or code,
a sound reproduction device such as headphones or earphones configured to apply auditory stimulus signals to the listener,
a response detector configured to detect and record listener responses to the presented auditory stimulus signals, and
a programmable sound generator configured to generate auditory stimulus signals in accordance with a plurality of signal parameters, wherein a processor of the test apparatus is configured to, by execution of the test program, execute steps of:
a) determining a two-dimensional boundary region surrounding an a priori estimated placement of the psychoacoustical threshold curve to form a predetermined two-dimensional response space comprising a positive response region at a first side of the a priori estimated psychoacoustical threshold curve and a negative response region at a second and opposite side of the a priori estimated psychoacoustical threshold curve,
b) optionally instructing the listener to detect a predetermined attribute/feature of the auditory stimulus signal,
c) determining a first parameter pair comprising a value of the first parameter and a value of the second parameter where the first parameter pair is situated in the positive response region,
d) presenting a first auditory stimulus signal in accordance with the first parameter pair to the listener through the sound reproduction device and recording listener's positive or negative detection of the predetermined attribute/feature of the first auditory stimulus,
e) presenting a subsequent auditory stimulus signal(s) to the listener in accordance with a subsequent parameter pair arranged adjacent to a former parameter pair in a first path direction through the two dimensional response space; wherein the first path direction heads towards the a priori estimated placement of the psychoacoustical threshold curve,
f) recording the listener's positive or negative detection of the predetermined attribute/feature of the subsequent auditory stimulus signal(s) and repeat steps e) and f) until a reversal detection in accordance with a predetermined detection reversal criterion is fulfilled in the first path direction or until the two-dimensional boundary region is reached,
g) select a subsequent parameter pair following the former parameter pair in a second path direction, differing from the first path direction and its reverse, in the predetermined two-dimensional response space, wherein the second path direction heads towards the a priori estimated placement of the psychoacoustical threshold curve,
h) presenting a subsequent auditory stimulus signal in accordance with the subsequent parameter pair to the listener and recording the listener's positive or negative detection of the predetermined attribute/feature of the subsequent auditory stimulus signal,
i) repeat step h) until a reversal detection in accordance with the predetermined detection reversal criterion is fulfilled in the second path direction or until the two-dimensional boundary region is reached,
j) repeating steps e), f), g), h) and i) one or more times to transverse and record a stimuli path through the predetermined two-dimensional response space extending forth and back across the psychoacoustical threshold curve,
k) determining the psychoacoustical threshold curve based on at least a subset of the recorded parameter pairs indicating the stimuli path through the predetermined two-dimensional response space.

16. (canceled)

17. A method of determining a psychoacoustical threshold curve according to claim 2, wherein the predetermined two-dimensional response space comprises a predetermined two-dimensional parameter grid structure comprising a plurality of parameter pairs comprising respective values of the first and second parameters.

18. A method of determining a psychoacoustical threshold curve according to claim 2, wherein the predetermined detection reversal criterion comprises:
identifying an initial response reversal in the first path direction or an initial response reversal in the second path direction,
selecting the subsequent parameter pair in opposite direction of the former parameter pair, and
present the subsequent auditory stimulus signal in accordance with the subsequent parameter pair.

19. A method of determining a psychoacoustical threshold curve according to claim 2, wherein the predetermined detection reversal criterion comprises:
identifying an initial detection reversal in the first path direction or an initial detection reversal in the second path direction,
repeating the presentation of the auditory stimulus signal that led to the initial detection reversal,
if the reversal detection is confirmed then proceed to step e) or step g) to proceed in an opposite path direction to a current direction; or
if the reversal detection is denied then determine a subsequent parameter pair arranged in the same path direction as the former parameter pair, and
present a subsequent auditory stimulus signal in accordance with the subsequent parameter pair.

20. A method of determining a psychoacoustical threshold curve according to claim 2, wherein the psychoacoustical threshold curve is either monotonically decreasing throughout the predetermined two-dimensional response space or monotonically increasing throughout the predetermined two-dimensional response space.

21. A method of determining a psychoacoustical threshold curve according to claim 2, comprising a measurement of an audiogram of the listener prior to performing step a) of claim 1.
